(19) United States
(12) Patent Application Publication
(10) Pub. No.: US 2006/0154908 A1
(43) Pub. Date: Jul. 13, 2006

(54) Co-administration of dehydroepiandrosterone (DHEA) congeners and other active agents for treating depression

(76) Inventors: Dinesh Patel, Salt Lake City, UT (US); David Vollmer, West Jordan, UT (US); Claire Daugherty, Salt Lake City, UT (US); John Gebhard, Salt Lake City, UT (US)

Correspondence Address: THORPE NORTH & WESTERN, LLP., 8180 SOUTH 700 EAST, SUITE 200, SANDY, UT, 84070, US

(21) Appl. No.: 11/342,276

(22) Filed: Jan. 26, 2006

Related U.S. Application Data
(63) Continuation-in-part of application No. 11/145,024, filed on Jun. 3, 2005.
(60) Provisional application No. 60/584,350, filed on Jun. 30, 2004.

(51) Int. Cl. A61K 31/57

(52) U.S. Cl. 514/171

(57) ABSTRACT

The present invention is drawn to compositions and methods for treating depression in a subject comprising co-administering therapeutically effective amounts of a DHEA congener and a second antidepressant agent to the subject, wherein the step of co-administrating is more effective in producing an anti-depressive effect compared to the administration of either the DHEA congener or the second antidepressant agent alone at their respective dosages.

This application is a continuation-in-part of U.S. patent application Ser. No.11/145,024, filed on Jun. 3, 2005, which claims the benefit of U. S. Provisional Application No. 60/584,350, filed on Jun. 30, 2004, both of which are incorporated herein by reference in their entireties.

### FIELD OF THE INVENTION

The present invention relates generally to methods and compositions for treating depression. More particularly, the present invention relates to the co-administration of dehydroepiandrosterone (DHEA) congener in combination with another pharmaceutically active agent to treat depression.

### BACKGROUND OF THE INVENTION

Clinical depression has gained increasing recognition as a serious public health issue worldwide. Imbalances in certain neurotransmitters, particularly serotonin, norepinephrine, and/or dopamine are believed to be an underlying cause of clinical depression. Currently there are three major categories of antidepressant drugs: selective serotonin reuptake inhibitors (SSRIs), tricyclics, and monoamine oxidase inhibitors. Often, patients will respond to one of these medicines (after a considerable time of dose titration) only to have the therapeutic effect wane. The reason for this ineffectiveness is not entirely understood.

There is growing evidence that improper immune responses, particularly with regards to the mechanisms of inflammation, can be related to the pathophysiology of clinical depression. Inflammation within a human subject is a common physiological response by the immune system to an injury or irritation, where the irritation can be caused by infectious, allergic, and/or chemical irritants. The immune response is carefully mediated through the release of both pro-inflammatory and anti-inflammatory cytokines. Pro-inflammatory cytokines include interleukin-1 (IL-1), interleukin-6 (IL-6), tumor necrosis factor alpha (TNF-α), and transforming growth factor beta (TGF-β). Dysregulated cytokine-signaling can reveal itself in the form of inflammatory disorders such as arthritis, inflammatory bowel disease, psoriasis, and asthma.

A recent and growing body of research has found a relationship between cytokine abnormalities and the pathophysiology of depression. In fact, it is well accepted that depression enhances the production of pro-inflammatory cytokines. Furthermore, inflammatory cytokines given to animals provoke symptoms similar to those seen in major depression. Therefore, depression can be caused by imbalanced cytokine secretion associated with activation of the immune system. The fact that immune system-derived cytokines influence the function of both neural and endocrine systems suggests that this is a plausible theory.

The theory that depression is worsened, directly or indirectly, by inflammation is supported by considerable clinical evidence showing a link between depressive symptoms and higher levels of pro-inflammatory cytokines. Pro-inflammatory mediators such as TNF-α, IL-1β, and IL-6 are increased in depressed patients as opposed to those that are not afflicted.

There is also considerable documented evidence that the endocrine system is disturbed in patients suffering from depression. Therefore, therapies that include enhancing (or decreasing) the levels of one or more relevant neurohormones can be advantageous in treating depression. Hence, the development of unique and novel pharmaceutical treatments under this auspice would be desirable.

### SUMMARY OF THE INVENTION

Briefly, and in general terms, the invention is directed to methods and compositions for treating depression. The method can comprise co-administering a dehydroepiandosterone (DHEA) congener and a-second anti-depressant agent to a subject. Co-administration of DHEA and a second anti-depressant agent in accordance with the invention typically provides a more effective anti-depressive than administration of either agent alone at their respective dosages.

In another embodiment, the present invention also provides methods for lessening adverse side effects that a subject can experience from taking antidepressant drugs, said methods comprising co-administering therapeutically effective amounts of a DHEA congener and the antidepressant drug to the subject.

In another embodiment, a composition for the treatment of depression in a subject can comprise a DHEA congener, a second antidepressant agent, and a carrier. The composition can be formulated to be more effective in producing an anti-depressive effect compared to the administration of either the DHEA congener or the second antidepressant agent alone at their respective dosages.

Additional features and advantages of the invention will be apparent from the detailed description which illustrates, by way of example, features of the invention.

### DETAILED DESCRIPTION OF PREFERRED EMBODIMENT(S)

Before particular embodiments of the present invention are disclosed and described, it is to be understood that this invention is not limited to the particular process and materials disclosed herein as such can vary to some degree. It is also to be understood that the terminology used herein is used for the purpose of describing particular embodiments only and is not intended to be limiting, as the scope of the present invention will be defined only by the appended claims and equivalents thereof.

The singular forms “a,” “an,” and “the” include plural referents unless the context clearly dictates otherwise. Thus, for example, reference to “a drug” includes reference to one or more of such drugs.

As used herein, the terms “formulation” and “composition” can be used interchangeably and refer to a combination of a pharmaceutically active agents, such as a DHEA congener formulated with one or more additional antidepressant agent(s). The terms “drug,” “active agent,” “bioactive agent,” “pharmaceutically active agent,” and “pharmaceutical,” can also be used interchangeably to refer to an agent or substance that has measurable specified or selected physiological activity when administered to a subject in an effective amount. When used to describe an agent or an effect, the terms “antidepressant” or “anti-depressive,” can refer to the *effect of lessening the symptoms of clinical depression in a subject. The term can also refer to the effect of inhibiting or lowering biochemical factors in the body of a subject that produce or contribute to the symptoms of clinical depression exhibited by the subject. These terms of art are well known in the pharmaceutical and medicinal arts.

As used herein, “administration,” and “administering” refer to the manner in which a drug, formulation, or composition is introduced into the body of a subject. Administration can be accomplished by various art-known routes such as oral, parenteral, transdermal (including transmucosal), inhalation, implantation, etc. Thus, an oral administration can be achieved by swallowing, chewing, or sucking an oral dosage form comprising active agent(s). Parenteral administration can be achieved by injecting a drug composition intravenously, intra-arterially, intramuscularly, intrathecally, or subcutaneously, etc. Transdermal administration can be accomplished by applying, pasting, rolling, attaching, pouring, pressing, rubbing, etc., of a transdermal preparation onto a skin surface. Transmucosal administration can be accomplished by bringing the composition into contact with any accessible mucous membrane for an amount of time sufficient to allow absorption of a therapeutically effective amount of the composition. Examples of transmucosal administration are: inserting a suppository into the rectum or vagina; by placing the composition on the oral mucosa, such as inside the cheek, on the tongue, or under the tongue; inhaling a vapor, mist, or aerosol into the nasal passage. These and additional methods of administration are well known in the art.

The term “co-administering,” “co-administration,” or “co-administer” refers to the administration of a DHEA congener with a second antidepressant agent. Both the DHEA congener and the second antidepressant agent can be administered simultaneously or immediately consecutively. Co-administration does not require the DHEA congener and the second anti-inflammatory agent to be administered by the same route. As such, each can be administered independently or as a common dosage form.

The terms “effective amount,” and “sufficient amount” can be used interchangeably and refer to an amount of an ingredient which, when included in a composition, is sufficient to achieve an intended compositional or physiological effect. Thus, a “therapeutically effective amount” refers to a non-toxic, but sufficient amount of an active agent, to achieve therapeutic results in treating a condition for which the active agent is known to be effective. Various biological factors can affect the ability of a substance to perform its intended task. Therefore, an “effective amount” or a “therapeutically effective amount” can be dependent on such biological factors. Further, while the achievement of therapeutic effects can be measured by a physician or other qualified medical personnel using evaluations known in the art, it is recognized that individual variation and response to treatments can make the achievement of therapeutic effects a subjective decision. In some instances, a “therapeutically effective amount” of a drug can achieve a therapeutic effect that is measurable by the subject receiving the drug. The determination of an effective amount is well within the ordinary skill in the art of pharmaceutical, medicinal, and health sciences.

As used herein, the terms “inhibit” or “inhibiting” refers to the process of holding back, suppressing, or restraining so as to block, prevent, limit, or decrease a rate of action or function. The use of the term is not to be misconstrued to be only of absolute prevention, but can be a referent of from any incremental step of limiting or reducing a function to the full and absolute prevention of the function.

As used herein, “reduce” or “reducing” refers to the process of decreasing, diminishing, or lessening, as in extent, amount, or degree of that which is reduced. The use of the term with respect to adverse side effects can mean resulting in reduced severity of a particular side effect, as well as a decrease in the number of different side effects suffered. Additionally, the use of the term can include from any minimal decrease to absolute abolishment of a physiological process or effect.

As used herein, “treat,” “treatment,” or “treating” refers to the process or result of giving medical aid to a subject, where the medical aid can counteract a malady, a symptom thereof, or other related adverse physiological manifestation. Additionally, these terms can refer to the administration or application of remedies to a patient or for a disease or injury; such as a medicine or a therapy. Accordingly, the substance or remedy so applied, such as the process of providing procedures or applications, are intended to relieve illness or injury. Additionally, the term can be used for the procedure of preemptively acting to prevent the malady, a symptom thereof, or other related adverse physiological manifestation. As such, a treatment can be administered prior to the subject experiencing any symptoms so that the symptoms are not manifested in the subject.

As used herein, “carrier” or “inert carrier” refers to typical compounds or compositions used to carry active ingredients, such as polymeric carriers, liquid carriers, or other carrier vehicles with which a bioactive agent, such as a DHEA congener and/or other antidepressant agents, can be combined to achieve a specific dosage form. As a general principle, carriers do not substantially react with the bioactive agent in a manner which substantially degrades or otherwise adversely affects the bioactive agent or its therapeutic potential.

As used herein, “subject” refers to an animal, such as a mammal, that can benefit from the administration of an inflammation reducing drug, a combination of drugs, or a formulation; or from a method for treating depression recited herein. Most often, the subject will be a human.

The term “maintenance dose,” when used to describe a dose of an antidepressant agent to be administered to a subject, can refer to the daily dose of antidepressant agent needed to maintain a desired therapeutic effect gained from administration of an initial dosage level.

As used herein, “mc” or “micro” when used in combination with a unit of measurement denotes the standard unit to be divided by one million, or multiplied by 1×10⁻⁶. Accordingly, the prefix “micro,” which is well known by one or ordinary skill in the art can be referred herein by the abbreviation “mc.”

As used herein, “mg/kg” or any other mass unit divided by another mass unit when used to describe a drug dose or dosing regimen denotes the mass of drug delivered per mass of the subject being administered the drug. Such use of units when referring to pharmaceuticals and their associated doses is well known to one of ordinary skill in the art.

As used herein, “mg/m²” or any other mass unit divided by an area unit when used to describe a drug dose or dosing regimen denotes the mass of the drug delivered per surface area of the subject being administered the drug. The use of mass of drug per surface area of subject when referring to pharmaceuticals and their associated doses is well known to one of ordinary skill in the art.

As used herein, “enhance” or “enhancing” of an anti-depressive response refers to the interaction of two or more active agents or drugs so that their combined physiological effect is greater than the individual effect of either active agent when administered alone at the same dosage.

The term “about” when referring to a numerical value or range is intended to encompass the values resulting from experimental error that can occur when taking measurements.

As used herein, a plurality of items, structural elements, compositional elements, and/or materials can be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data can be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight range of about 1 wt % to about 20 wt % should be interpreted to include not only the explicitly recited concentration limits of 1 wt % to about 20 wt %, but also to include individual concentrations such as 2 wt %, 3 wt %, 4 wt %, and sub-ranges such as 5 wt % to 15 wt %, 10 wt % to 20 wt %, etc.

The term “dehydroepiandrosterone congener” or “DHEA congener” includes dehydroepiandrosterone (a.k.a. DHEA and (3β)-3-hydroxyandrost-5-en-17-one), derivatives of DHEA, metabolites of DHEA, metabolites of DHEA derivatives; salts of DHEA, salts of DHEA derivatives, etc. DHEA, generally, is a weak androgen that serves as the primary precursor in the biosynthesis of both androgens and estrogens. Typically, a DHEA congener used in accordance with embodiments of the present invention is in a pharmaceutically acceptable form.

In further detail regarding DHEA, an endogenous neurosteroid produced by the adrenal glands, this composition is a precursor to over 50 other hormones in the body. DHEA has a number of physiological actions in mammals, including inhibiting inflammation. The exact mechanism underlying this anti-inflammatory effect is not well understood. However, it is believed to disrupt the inflammatory signal transduction pathway at various points. Most significantly, DHEA is known to lower the serum levels of pro-inflammatory cytokines such as TNF-α, IL-1β, and IL-6.

There appears to be a connection between levels of these same cytokines and clinical depression. For example, these cytokines are often elevated in patients suffering from clinical depression as compared to those that are not afflicted. Also, depression is known to be exacerbated by inflammation. These and similar phenomena indicate that depression can be caused either directly or indirectly by inflammation and pro-inflammatory cytokines. Therefore, administering DHEA to a subject can provide an antidepressant effect in that subject by suppressing pro-inflammatory cytokines.

The neurotransmitter serotonin has proven to be a significant factor in the neurobiology of depression. Serotonin (5-hydroxytryptamine, 5-HT) mediates a wide range of physiological functions by interacting with multiple receptors. Of particular interest are the sigma serotonergic receptors, of which there are at least two: σ-1 and σ-2. These intracellular receptors are mainly located on the endoplasmic reticulum of neurons, and are expressed in specific regions of the brain, such as layers of the cortex, hippocampus, hypothalamic nuclei, substantia nigra and Purkinje cells in the cerebellum. The most commonly utilized class of antidepressants, the selective serotonin reuptake inhibitors (SSRIs), have high affinity for σ-1 receptors. Steroid hormones, including the neurosteroid DHEA, have also been found to bind σ-1 receptors. Therefore, DHEA can also provide an antidepressant effect to a subject by its interaction with σ-1 receptors.

The benefits associated with administration of SSRIs, can be offset somewhat by the occurrence of various side effects in many subjects, including sexual dysfunction (such as abnormal ejaculation and priapism), nausea, headache, nervousness, insomnia, dry mouth, diarrhea, and anorexia. It is believed that many of these effects can be mediated by the binding of SSRIs with other serotonergic receptors besides σ-1. However, because DHEA does not interact with these other receptors, it can provide antidepressant effects from binding σ-1 receptors with a lower occurrence of adverse side effects as compared to SSRIs or other antidepressant agents.

These benefits can be enhanced (or coadministration of a DHEA congener can provide benefits to the anti-depressant) by co-delivering a second antidepressant agent with a DHEA congener, which will also be described in more detail below. This being stated, according to particular embodiments of the present invention, the second antidepressant agent can be a selective serotonin reuptake inhibitor. According to another particular embodiment, the second antidepressant agent can be a monoamine oxidase inhibitor. According to yet another particular embodiment, the second antidepressant agent can be a tricyclic antidepressant. According to yet another particular embodiment, the second antidepressant agent can be a tetracyclic antidepressant. Other anti-depressants can also be used.

With this brief description in mind, the present invention provides methods for treating depression by co-administering a DHEA congener and a second antidepressant agent, or lessening the negative effects of anti-depressant agents by co-delivering a DHEA congener. The invention also provides compositions for treating depression comprising a DHEA congener and a second antidepressant agent.

A) Dehydroepiandrosterone Congeners

As stated, a DHEA congener includes DHEA (3β)-3-hydroxyandrost-5-en-17-one), derivatives of DHEA, metabolites of DHEA, metabolites of DHEA derivatives, salts of DHEA, salts of DHEA derivatives, etc. Typically, a DHEA congener used in accordance with embodiments of the present invention is in a pharmaceutically acceptable form. Examples of DHEA congeners include, but are not limited to, compounds having the general formula 1, and their metabolites and pharmaceutically acceptable salts thereof: 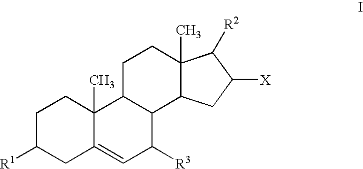
wherein

X is H or halogen;

R¹, R² and R³ are independently ═O, —OH, —SH, H, halogen, pharmaceutically acceptable esters, pharmaceutically acceptable thioesters, pharmaceutically acceptable ethers, pharmaceutically acceptable thioethers, pharmaceutically acceptable inorganic esters, pharmaceutically-acceptable monosaccharides, disaccharides or oligosaccharides, spirooxiranes, spirothiranes, —OSO₂R⁴ or —OPOR⁴R⁵;

R⁴ and R⁵ are independently —OH, pharmaceutically acceptable esters or pharmaceutically acceptable ethers.

Suitable metabolites of DHEA include, but are not limited to, dehydroepiandrosterone sulfate, 16α-hydroxydehydroepiandrosterone, 16α-hydroxyandrost-4-ene-3,17dione, androst-4-ene-3,17 dione, 7α-hydroxyandrostenedione, 7α-hydroxytestosterone.

Further examples of DHEA congeners, include but are not limited to, compounds having the general formulas II and III, and their metabolites and pharmaceutically acceptable salts thereof: 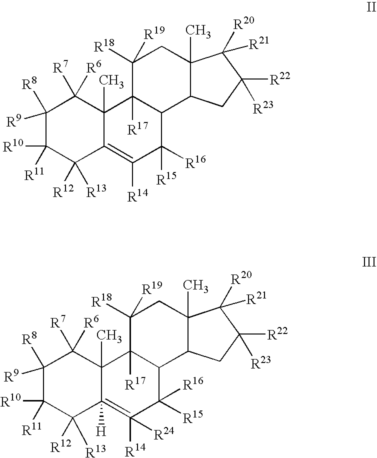
wherein
R⁶, R⁷, R⁸, R⁹, R¹¹, R¹², R¹³, R¹⁴, R¹⁵, R¹⁶, R¹⁷, R¹⁸, R¹⁹ and R²⁴ are independently H. ═OH, halogen, C₁₋₁₀ alkyl or C₁₋₁₀ alkoxy;
R¹⁰ is H, —OH, halogen, C₁₋₁₀ alkyl, or C₁₋₁₀ alkoxy;
R²⁰ is (1) H, halogen, C₁₋₁₀ alkyl or C₁₋₁₀ alkoxy when R²¹ is —C(O)OR²⁵ or
(2) H, halogen, OH or C₁₋₁₀ alkyl when R²¹ is H, halogen, OH or C₁₋₁₀ alkyl or
(3) H, halogen, C₁₋₁₀ alkyl, C₁₋₁₀ alkenyl, C₁₋₁₀ alkynyl, formyl, C₁₋₁₀ alkanoyl or epoxy when R²¹ is OH; or
R²⁰ and R²¹ taken together are ═O;
R²² and R²³ are independently (1) H, —OH, halogen, C₁₋₁₀ alkyl or C₁₋₁₀ alkoxy when R²¹ is H, OH, halogen, C₁₋₁₀ alkyl or —C(O)OR²⁵ or (2) H, (C₁₋₁₀ alkyl)ₙamino, (C₁₋₁₀ alkyl)ₙamino-C₁₋₁₀ alkyl, C₁₋₁₀ alkoxy, hydroxy-C₁₋₁₀ alkyl, C₁₋₁₀ alkoxy-C₁₋₁₀ alkyl, (halogen)ₘ-C₁₋₁₀ alkyl, C₁₋₁₀ alkanoyl, formyl, C₁₋₁₀ carbalkoxy or C₁₋₁₀ alkanoyloxy when R²⁰ and R²¹ taken together are ═O; or
R²² and R²³ taken together are ═O or taken together with the carbon to which they are attached form a 3-6 member ring containing 0 or 1 oxygen atom; or
R²⁰ and R²² taken together with the carbons to which they are attached form an epoxide ring;
R²⁵ is H, (halogen)ₘ-C₁₋₁₀ alkyl or C₁₋₁₀ alkyl;
n is 0, 1 or 2;
m is 1, 2 or 3; and
physiologically acceptable salts thereof, with the provisos that
(a) R¹⁰ is not H, halogen, or C₁₋₁₀ alkoxy when R⁶, R⁷, R⁸, R⁹, R¹¹, R¹², R¹³, R¹⁴, R¹⁵, R¹⁷, R¹⁸, R¹⁹ and R²² are H and R¹⁶ is H, halogen, OH or C₁₋₁₀ alkoxy and R²³ is H or halogen and R²⁰ and R²¹ taken together are ═O; and
(b) R¹⁰ is not H, halogen, or C₁₋₁₀ alkoxy when R⁶, R⁷, R⁸, R⁹, R¹¹, R¹², R¹³, R¹⁴, R¹⁵, R¹⁷, R¹⁸, R¹⁹ and R²² are H and R¹⁶ is H, halogen, OH or C₁₋₁₀ alkoxy and R²³ is H or halogen and R²⁰ is H and R²¹ is H, OH or halogen.

The compounds represented by the general formula I exist in many stereoisomers and the formula is intended to encompass the various stereoisomers. Examples of suitable DHEA congeners of Formula I include compounds in which:

(1) R² is ═O, R³ and X are each H and R¹ is ═O, —OH, pharmaceutically acceptable esters thereof, pharmaceutically acceptable ethers thereof or pharmaceutically acceptable salts;

(2) R² is ═O, R³ is H, X is halogen and R¹ is ═O, —OH, pharmaceutically acceptable esters thereof, pharmaceutically acceptable ethers thereof or pharmaceutically acceptable salts;

(3) R² is ═O, R³ and X are each H and R¹ is —SH, pharmaceutically acceptable thioesters thereof, pharmaceutically acceptable thioethers thereof or pharmaceutically acceptable salts;

(4) R² is ═O, R³ is H, X is halogen and R¹ is —SH, pharmaceutically acceptable thioesters thereof, pharmaceutically acceptable thioethers thereof or pharmaceutically acceptable salts;

(5) R² is ═O, X is H and R¹ and R³ are independently ═O, —OH, pharmaceutically acceptable esters thereof, pharmaceutically acceptable ethers thereof or pharmaceutically acceptable salts;

(6) R² is ═O, X is halogen and R¹ and R³ are independently ═O, —OH, pharmaceutically acceptable esters thereof, pharmaceutically acceptable ethers thereof or pharmaceutically acceptable salts;

(7) R² is ═O, X is H and R¹ and R³ are independently —SH, pharmaceutically acceptable thioesters thereof, pharmaceutically acceptable thioethers thereof or pharmaceutically acceptable salts;.

(8) R² is ═O, X is halogen and R¹ and R³ are independently —SH, pharmaceutically acceptable thioesters thereof, pharmaceutically acceptable thioethers thereof or pharmaceutically acceptable salts;

(9) R² is —OH, R³ and X are each H and R¹ is ═O, —OH, pharmaceutically acceptable esters thereof, pharmaceutically acceptable ethers thereof or pharmaceutically acceptable salts;

(10) R²is —OH, R³is H, X is halogen and R¹ is ═O, —OH, pharmaceutically acceptable esters thereof, pharmaceutically acceptable ethers thereof or pharmaceutically acceptable salts;

(11) R² is —OH, R³ and X are each H and R¹ is —SH, pharmaceutically acceptable thioesters thereof, pharmaceutically acceptable thioethers thereof or pharmaceutically acceptable salts;

(12) R²is —OH, R³is H, X is halogen and R¹ is —SH, pharmaceutically acceptable thioesters thereof, pharmaceutically acceptable thioethers thereof or pharmaceutically acceptable salts;

(13) R² is —OH, X is H and R¹ and R³ are independently ═O, —OH, pharmaceutically acceptable esters thereof, pharmaceutically acceptable ethers thereof or pharmaceutically acceptable salts;

(14) R² is —OH, X is halogen and R¹ and R³ are independently ═O, —OH, pharmaceutically acceptable esters thereof, pharmaceutically acceptable ethers thereof or pharmaceutically acceptable salts;

(15) R² is —OH, X is H and R¹ and R³ are independently —SH, pharmaceutically acceptable thioesters thereof, pharmaceutically acceptable thioethers thereof or pharmaceutically acceptable salts;

(16) R² is —OH, X is halogen and R¹ and R³ are independently —SH, pharmaceutically acceptable thioesters thereof, pharmaceutically acceptable thioethers thereof or pharmaceutically acceptable salts;

(17) R² is —SH, R³ and X are each H and R¹ is ═O, —OH, pharmaceutically acceptable esters thereof, pharmaceutically acceptable ethers thereof or pharmaceutically acceptable salts;

(18) R² is —SH, R³ is H, X is halogen and R¹ is ═O, —OH, pharmaceutically acceptable esters thereof, pharmaceutically acceptable ethers thereof or pharmaceutically acceptable salts;

(19) R² is —SH, R³ and X are each H and R¹ is —SH, pharmaceutically acceptable thioesters thereof, pharmaceutically acceptable thioethers thereof or pharmaceutically acceptable salts;

(20) R² is —SH, R³ is H, X is halogen and R¹ is —SH, pharmaceutically acceptable thioesters thereof, pharmaceutically acceptable thioethers thereof or pharmaceutically acceptable salts;

(21) R² is —SH, X is H and R¹ and R³ are independently ═O, —OH, pharmaceutically acceptable esters thereof, pharmaceutically acceptable ethers thereof or pharmaceutically acceptable salts;

(22) R² is —SH, X is halogen and R¹ and R³ are independently ═O, —OH, pharmaceutically acceptable esters thereof, pharmaceutically acceptable ethers thereof or pharmaceutically acceptable salts;

(23) R² is —SH, X is H and R¹ and R³ are independently —SH, pharmaceutically acceptable thioesters thereof, pharmaceutically acceptable thioethers thereof or pharmaceutically acceptable salts;

(24) R² is —SH, X is halogen and R¹ and R³ are independently —SH, pharmaceutically acceptable thioesters thereof, pharmaceutically acceptable thioethers thereof or pharmaceutically acceptable salts;

(25) X is H and R¹ is ═O, —OH, pharmaceutically acceptable esters thereof, pharmaceutically acceptable ethers thereof or pharmaceutically acceptable salts, R² and R³ are independently ═O, —OH, a sugar residue, pharmaceutically acceptable esters thereof, pharmaceutically acceptable ethers thereof or pharmaceutically acceptable salts, wherein at least one of R² and R³ is a sugar residue;

(26) X is halogen and R¹ is ═O, —OH, pharmaceutically acceptable esters thereof, pharmaceutically acceptable ethers thereof or pharmaceutically acceptable salts, R² and R³ are independently ═O, —OH, a sugar residue, pharmaceutically acceptable esters thereof, pharmaceutically acceptable ethers thereof or pharmaceutically acceptable salts, wherein at least one of R² and R³is a sugar residue;

(27) X is H, R¹ is ═O or —OH, and R² and R³ are independently ═O, —OH, pharmaceutically acceptable inorganic esters thereof or pharmaceutically acceptable salts, wherein at least one of R² and R³ is an inorganic ester; and/or (28) X is halogen R¹ is ═O or —OH, and R² and R³ are independently ═O, —OH, pharmaceutically acceptable inorganic esters thereof or pharmaceutically acceptable salts, wherein at least one of R² and R³ is an inorganic ester.

Pharmaceutically acceptable esters or thioesters include, but are not limited to, esters or thioesters of the formula —OOCR or —SOCR, wherein R is a pharmaceutically acceptable alkyl, alkenyl, aryl, alkylaryl, arylalkyl, sphingosine or substituted sphingolipid groups, such as propionate, enanthate, cypionate, succinate, decanoate and phenylpropionate esters.

Pharmaceutically acceptable ethers or thioethers include, but are not limited to, ethers or thioethers of the formula —OR or —SR, wherein R is as defined above or enol, or -OR is an unsubstituted or substituted spirooxirane or —SR is a spirothiane.

Suitable sugar residues can include, but are not limited to monosaccharides, disaccharides, and oligosaccharides, such as a glucuronate.

Pharmaceutically acceptable inorganic esters include, but are not limited to, esters of the formula —OSO₂R⁴ or —OPOR⁴R⁵, wherein R⁴ and R⁵ are independently —OH, pharmaceutically acceptable esters, pharmaceutically acceptable ethers or pharmaceutically acceptable salts.

Some DHEA congeners, such as the compounds of general formulas I, II, and III, can be synthesized as described in U.S. Pat. Nos. 4,898,694; 5,001,119; 5,028,631; and 5,175,154, which are all incorporated herein by reference. The compounds represented by the general formulas II and III exist in many stereoisomers and these formulas are intended to encompass the various stereoisomers. Examples of representative compounds, which fall within the scope of general formulas II and III, include the following: 5α-androstan-17-one; 16α-fluoro-5α-androstan-17-one; 3β-methyl-5α-androsten-17-one; 16β-fluoro-5α-androstan-17-one; 17β-bromo-5-androsten-16-one; 17β-fluoro-3β-methyl-5-androsten-16-one; 17α-fluoro-5α-androstan-16-one; 3β-hydroxy-5-androsten-17-one; 17α-methyl-5α-androstan-16-one; 16α-methyl-5-androsten-17-one; 3β,16α-dimethyl-5-androsten-17-one; 3β,17α-dimethyl-5-androsten-16-one; 16α-hydroxy-5-androsten-17-one; 16α-fluoro-16β-methyl-5-androsten-17-one; 16α-methyl-5a-androstan-17-one; 16-dimethylaminomethyl-5α-androstan-17-one; 16β-methoxy-5-androsten-17-one; 16α-fluoromethyl-5-androsten-17-one; 16-methylene-5-androsten-17-one; 16-cyclopropyl-5α-androstan-17-one; 16-cyclobutyl-5-androsten-17-one; 16-hydroxymethylene-5-androsten-17-one; 3α-bromo-16α-methoxy-5-androsten-17-one; 16-oxymethylene-5-androsten-17-one; 3β-methyl-16ξ-trifluoromethyl-5α-androstan-17-one; 16-carbomethoxy-5-androsten-17-one; 3β-methyl-16β-methoxy-5α-androstan-17-one; 3β-hydroxy-16α-dimethylamino-5-androsten-17-one; 17α-methyl-5-androsten-17β-ol; 17α-ethynyl-5α-androstan-17β-ol; 17β-formyl-5α-androstan-17β-ol; 20,21-epoxy-5α-pregnan-17α-ol; 3β-hydroxy-20,21-epoxy-5α-pregnan-17α-ol; 16α-fluoro-17α-ethenyl-5-androsten-17β-ol; 16α-hydroxy-5-androsten-17α-ol; 16α-methyl-5α-androstan-17α-ol; 16α-methyl-16β-fluoro-5α-androstan-17α-ol; 16α-methyl-16β-fluoro-3-hydroxy-5-androsten-17α-ol; 3β,16β-dimethyl-5-androsten-17β-ol; 3β,16,16-trimethyl-5-androsten-17β-ol; 3β,16,16-trimethyl-5-androsten-17-one; 3β-hydroxy-4α-methyl-5-androsten-17α-ol; 3β-hydroxy-4α-methyl-5-androsten-17-one; 3α-hydroxy-1α-methyl-5-androsten-17-one; 3α-ethoxy-5α-androstan-17β-ol; 5α-pregnan-20-one; 3β-methyl-5α-pregnan-20-one; 16α-methyl-5-pregnen-20-one; 16α-methyl-3β-hydroxy-5-pregnen-20-one; 17α-fluoro-5-pregnen-20-one; 21 -fluoro-5α-pregnan-20-one; 17α-methyl-5-pregnen-20-one; 20-acetoxy-cis-17(20)-5α-pregnene; and 3α-methyl-16,17-epoxy-5-pregnen-20-one, for example.

In one aspect of the present invention, a DHEA congener and a second antidepressant agent can be co-administered to a subject in an amount that results in a therapeutic effect, thereby aiding in treating and/or preventing depression in a subject. The dose of the DHEA congener administered is selected to achieve DHEA or DHEA equivalent blood levels greater than normal endogenous DHEA blood levels of the subject. Normal endogenous blood levels of DHEA can be less than 20 ng/mL. Accordingly, peak blood levels of DHEA or DHEA equivalent can be greater than about 20 ng/mL, or as desired for a specific therapeutic effect. In one aspect, suitable doses can be selected to achieve a peak blood level of DHEA or DHEA equivalent that can be in the range from about 20 ng/mL to about 100 mg/mL, or in the range from about 30 ng/mL to about 10 mg/mL. Additionally, the doses administered to a subject can be in an amount to achieve DHEA blood levels in the subject from about 100 ng/mL to about 1 mg/mL, from 100 ng/mL to about 100 pg/mL, and/or from about 100 ng/mL to about 10 μg/mL. Persons having skill in the relevant art will understand the need to titrate DHEA levels in combination with the second antidepressant agent so as to achieve the desired therapeutic effect. In one embodiment of the invention, more effective dosing of DHEA for a given subject can be achieved by first determining the subject's serum concentration of DHEA or a DHEA equivalent, such as DHEA sulfate. DHEA levels can be measured from blood samples or saliva by techniques that are known to those skilled in the art.

In accordance with the methods of the present invention, a DHEA congener can be administered as a part of a regimen to aid in treating depression in a subject having need of such treatment. In one aspect, a DHEA congener can be administered in a dosing regimen that includes providing from about 10 mg to about 3000 mg per day of the DHEA congener to the subject. In a more particular aspect, a DHEA congener can be administered in a dosing regimen that includes providing from about 100 mg to about 1500 mg of the DHEA congener. These dosages can be administered once a day, or administered in two or three smaller dosages throughout the day in accordance with the present invention. For example, a subject who is to receive 1500 mg of DHEA daily can be administered one 1,500 mg dose per day, or two 750 mg doses, or three 500 mg doses.

B) Antidepressant Agents

The present invention provides for co-administration of DHEA with a second antidepressant agent for the-treatment of depression. A number of antidepressant drugs have been developed, exhibiting various modes of action and levels of efficacy. Safe and efficacious use of these drugs often involves administering them under certain dosing regimens. The dose administered to the subject at a given time can depend on the stage of the dosing regimen. Under one exemplary dosing regimen, the subject is administered a drug at an initial dosage amount, which is gradually increased while the subject is monitored for signs of intolerance, until the desired therapeutic effect is observed. For the remainder of the dosing regimen, the subject can then be administered the lowest dose needed to maintain the desired therapeutic effect. Other dosing regimens can involve i) continuing a known dosing regimen and co-administering a DHEA congener to reduce side effects; ii) setting a dosing regimen based on physical characteristics of the subject; iii) or the like.

The antidepressants listed below are exemplary of the large array of antidepressants that can be used in accordance with embodiments of the present invention. The structure of the dosing regimes available for each is known to those having skill in the medical and pharmaceutical arts, but can be modified or remain the same when co-delivering a DHEA congener. Each of the following exemplary dosing regimen embodiments for the anti-depressants are to be understood as dosing regimens for co-delivery with a DHEA congener, such as those DHEA congener dosing regimens set forth above. For the drugs listed below and for others, the specific dosing regime to be administered to a subject depends upon a number of factors, including the severity of the subject's symptoms and the subject's tolerance for the drug in question. This regime can be ascertained by those having skill in the art by using known techniques for assessing these factors. However, it should be noted that typically, each dosage range given is exemplary only, and these dosages can be administered either as a single dose, as a sustained release dose, or in multiple doses over a period of a day (or other unit of time as specified), whether such a dosing regime is specifically specified or not as being so modifiable.

i) Selective Serotonin Reuptake Inhibitors

As noted above,; the neurotransmitter serotonin has been implicated as having a significant role in the neurophysiology of depression. Shortly after serotonin is released from a presynaptic neuron into the synaptic cleft separating it from a postsynaptic neuron, reuptake pumps work to pull serotonin molecules back into the presynaptic neuron for repackaging and eventual re-release. Selective serotonin reuptake inhibitors (SSRIs) inhibit the action of these pumps so that'serotonin remains in the cleft longer to allow further binding with postsynaptic receptors. SSRIs are a commonly utilized class of antidepressant drugs. In certain embodiments of the present invention, the second antidepressant agent administered to the subject can be an SSRI. The following are examples of SSRis that can be co-administered with DHEA according to particular aspects of these embodiments.

Citalopram can be administered in an amount from 2 mg to 90 mg, or alternatively from 20 mg to 60 mg, daily in accordance with the present invention.

Escitalopram oxalate can be administered in an amount from 1 mg to 30 mg, or from 10 mg to 20 mg, in accordance with the present invention. Escitalopram oxalate also helps alleviate anxiety.

Paroxetine can be administered in accordance with the present invention in an amount from 2 mg to 60 mg, or alternatively from 20 mg to 40 mg, per day. Paroxetine can also be administered in a controlled-release formulation, where the daily amount of release can be from 2 mg to 75 mg, or alternatively from 12.5 mg to 50 mg. Paroxetine can also be administered to treat panic disorder, obsessive-compulsive disorder (OCD), social anxiety disorder, generalized anxiety disorder, and post-traumatic stress disorder (PTSD).

Fluoxetine can be administered in an amount from 2 mg to 120 mg, or alternatively from 20 mg to 80 mg, per day. This drug can also be used to treat OCD, bulimia, and panic disorder.

Fluvoxamine maleate can be administered in an amount from 5 mg to 450 mg, or alternatively from 50 mg to 300 mg, per day. In a more particular aspect, fluvoxamine maleate can be administered in an initial dose of 50 mg, and then the dose can be increased by 50 mg every 4 to 7 days. If the daily dose exceeds 100 mg under this regime, fluvoxamine maleate can be given in equally divided doses, or a larger dose can be given at bedtime. Fluvoxamine maleate can also be administered to treat OCD.

Sertraline can be administered in an amount from 5 mg to 150 mg, or alternatively from 50 mg to 100 mg, per day. In a more particular aspect, the dose of sertraline can be titrated upward over a period of time.

ii) Monoamine Oxidase Inhibitors

Monoamine oxidase inhibitors (MAOIs) facilitate the action of monoamine neurotransmitters by inhibiting their breakdown by the enzyme monoamine oxidase. As a result, monoamines released by a cell remain in the synaptic cleft longer, and are available for binding a postsynaptic cell for a longer time. In certain embodiments of the present invention, the second antidepressant agent administered to the subject can be an MAOI. The following are examples of MAOIs that can therefore be co-administered with DHEA congeners according to particular aspects of those embodiments.

Phenelzine can be administered in an amount from 5 mg to 150 mg, or alternatively from 45 mg to 90 mg, per day. In a more particular aspect, the daily dose of phenelzine can be divided into three smaller doses.

Tranylcypromine can be administered in an amount from 3 mg to 90 mg, or alternatively from 30 mg to 60 mg, per day. In a more particular aspect, the daily dose of tranylcypromine can be divided into two or more doses. In another aspect, the dose of tranylcypromine can be increased by 10 mg per day increments every one to three weeks.

Isocarboxazid can be administered in an amount from 2 mg to 90 mg, or alternatively from 20 mg to 60 mg, per day. In a more particular aspect, isocarboxazid can be administered initially in two 10 mg doses per day, adding a 10 mg per day dose every two to four days to achieve a dosage of four tablets daily (40 mg) by the end of the first week of treatment. Dosage can then be increased by increments of up to 20 mg/week, if needed and tolerated, to a maximum recommended dosage of 60 mg/day.

iii) Tricyclic Antidepressants

Tricyclic antidepressants (TCAs) constitute a group of drugs that have been popular since the 1950s in treating depression. TCAs are believed to alleviate depression by increasing the levels of serotonin and norepinephrine in the brain, at least in part by inhibiting reuptake of these transmitters. In certain embodiments of the present invention, the second antidepressant agent administered to the subject can be a TCA. The following are examples of TCAs that can be co-administered with DHEA according to particular aspects of the present invention.

Amitriptyline can be administered in an amount from 3 mg to 450 mg, or alternatively from 25 mg to 300 mg, per day. In a more particular aspect, the dose can be administered at the subject's bedtime. In another aspect, amitriptyline can be administered at an initial dose of from 25 mg to 100 mg per day, then the daily dose is increased gradually to an amount from 50 mg to 300 mg per day.

Amoxapine can be administered in an amount from 10 mg to 750 mg, or alternatively from 100 mg to 500 mg, per day. In a more particular aspect, the daily dose amoxapine can be divided into two or three daily doses. In still more particular aspect, the daily dose of amoxipine can be increased over a period of one week from two or three 50 mg doses per day to two or three 150 mg doses per day.

Clomipramine can be administered in an amount from 3 mg to 450 mg, or alternatively from 25 mg to 300 mg, per day. In a more particular aspect, clomipramine can be initially administered at a daily dose of 25 mg, with the daily dose increased by 25 mg increments at three- or four-day intervals up to a daily dose of 150 mg by the end of two weeks. Thereafter, the daily dose can be gradually increased over a period of several weeks to 200 mg, or up to 300 mg in severely depressed hospitalized subjects.

Desipramine can be administered in an amount from 3 mg to 450 mg, or alternatively from 25 mg to 300 mg, per day. In a more particular aspect, the daily dose of desipramine can be divided into two or more doses.

Doxepin can be administered in an amount from 3 mg to 450 mg, or alternatively from 25 mg to 300 mg, per day. In a more particular aspect, the dose can be administered at the subject's bedtime.

Imipramine can be administered in a daily amount from 3 mg to 450 mg, or alternatively from 25 mg to 300 mg. In a more particular aspect, the dose can be administered at the subject's bedtime. In another aspect, the daily dose of imipramine can be increased gradually from 25 mg per day to a therapeutically effective dose, which can be from 50 mg to 300 mg per day.

Nortryptyline can be administered in an amount from 3 mg to 225 mg, or alternatively from 25 mg to 150 mg, per day. In a more particular aspect, the daily dose of nortryptyline can be divided into from two to four doses.

Protriptyline can be administered to ambulatory patients in an amount from 2 mg to 45 mg, or alternatively from 15 mg to 30 mg. In one embodiment, as with any of the other embodiments, the dosages can be divided into two or more daily doses. Alternatively, protryptyline can be administered to hospitalized subjects in an amount from 30 mg to 60 mg per day in two or more doses.

Trimipramine can be administered in an amount from 5 mg to 300 mg, or alternatively from 50 mg to 200 mg, per day. Again, in one embodiment, as with any of the other embodiments, the dosages can be divided into two or more daily doses. In a more particular aspect, a daily dose of from 50 mg to 150 mg of trimipramine can be administered to maintain a therapeutic effect achieved by an earlier dosing regime with this drug.

iv) Tetracyclic Antidepressants

Tetracyclic antidepressant drugs are similar to tricyclic antidepressants in action and effect. Tetracyclic antidepressants are believed to cause less pronounced side effects than many TCAs. In certain embodiments of the present invention, the second antidepressant agent administered to the subject can be a tetracyclic antidepressant. The following are examples of tetracyclic antidepressants that can be co-administered with DHEA according to particular aspects of the present invention.

Maprotiline can be administered in an amount from 3 mg to 225 mg, or alternatively from 25 mg to 150 mg, per day. In a more particular aspect, this amount of maprotiline can be divided into two or three daily doses.

Mirtazapine can be administered in an amount from 2 mg to 70 mg, or alternatively from 15 mg to 45 mg, per day.

v) Other Antidepressants

There are a number of antidepressant drugs that do not fall into the above categories. Many inhibit the action of two or more neurotransmitters, including serotonin, dopamine, and norepinephrine. The following are examples of other antidepressants that can be co-administered with DHEA according to particular aspects of the present invention.

Bupropion can be administered -in an amount from 15 mg to 450 mg, or alternatively from 150 mg to 300 mg, per day.

Duloxetine can be administered in a dose of from 4 mg to 90 mg per day, or alternatively from 40 mg to 60 mg of duloxetine can be administered daily.

Nefazodone can be administered in an amount from 20 mg to 900 mg, or alternatively from 200 mg to 600 mg, per day. In a more particular aspect, the amount of nefazodone administered can be divided into two daily doses.

Trazodone can be administered in an amount from 5 mg to 600 mg, or alternatively from 50 mg to 400 mg, per day.

Venlafaxine can be administered in an amount from 4 mg to 350 mg, or alternatively from 35 mg to 225 mg, per day. In a more particular aspect, venlafaxine can be administered at a dose of 37.5 mg per day for one week, and then titrated upward until the desired therapeutic effect is achieved.

### EXAMPLES

The following examples illustrate the embodiments of the invention that are presently best known. However, it is to be understood that the following are only exemplary or illustrative of the application of the principles of the present invention. Numerous modifications and alternative compositions and methods can be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

A human subject experiencing symptoms of clinical depression is identified. The subject is administered 250 mg of DHEA together with 125 mg of nefazodone, both in tablet form, twice daily. The subject experiences a lessening in the severity of the symptoms, and this effect persists for the duration of this treatment.

### Example 2

A human subject experiencing symptoms of clinical depression is identified. The subject is administered DHEA together with citalopram, both in tablet form, once daily according to the dosing regime set forth in Table 1:

TABLE 1
Dosing regime for co-administration of DHEA and citalopram

Dose

| Day | DHEA (mg) | citalopram (mg) |
|---|---|---|
| 1 | 500 | 20 |
| 2 | 500 | 25 |
| 3 | 500 | 30 |
| 4 | 500 | 35 |
| 5 | 500 | 40 |
| 6+ | 500 | 40 |
| (maintenance) | | |

During the first five days of administration, the dose of citalopram is gradually increased until the subject experiences a lessening of symptoms, eventually achieving a desired symptom level or an absence of symptoms by Day 5. After Day 5, the subject is co-administered DHEA and citalopram at a steady daily dose to maintain the desired symptom level.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

What is claimed is:

1. A method of treating depression in a subject comprising co-administering therapeutically effective amounts of a DHEA congener and a second antidepressant agent to the subject, wherein said step of co-administering is by co-delivering the DHEA congener and the second antidepressant simultaneously or immediately consecutively, and wherein said co-administering is more effective in producing an anti-depressive effect compared to the administration of either the DHEA congener or the second antidepressant agent alone at their respective dosages.

2. A method as in claim 1, further comprising the preliminary step of determining the serum concentration of DHEA or DHEA equivalent in the subject.

3. A method as in claim 1, wherein the serum concentration is used to determine the dosage of delivery of the DHEA congener.

4. A method as in claim 1, wherein the DHEA congener is administered to the subject in an amount from 10 mg to 3000 mg per day.

5. A method as in claim 4, wherein the DHEA congener is administered to the subject in an amount from 100 mg to 1500 mg per day.

6. A method as in claim 1, wherein the co-administering step results in enhanced anti-depressive effect compared to the administration of either the DHEA congener or the second antidepressant agent alone at their respective dosages.

7. A method as in claim 1, wherein the DHEA congener and the second antidepressant agent are each administered to the subject by a route selected from the group consisting of oral, nasal, transdermal, parenteral, transmucosal, and transdermal.

8. A method as in claim 7, wherein the DHEA congener and the second antidepressant agent are administered to the subject by the same route.

9. A method as in claim 8, wherein the DHEA congener and the second antidepressant agent are administered together in a single dosage form.

10. A method as in claim 1, wherein the second antidepressant agent is a selective serotonin reuptake inhibitor.

11. A method as in claim 10, wherein the selective serotonin reuptake inhibitor is a member selected from the group consisting of citalopram, escitalopram oxalate, paroxetine, fluoxetine, fluvoxamine maleate, sertraline, and combinations thereof.

12. A method as in claim 1, wherein the second antidepressant agent is a monoamine oxidase inhibitor.

13. A method as in claim 12, wherein the monoamine oxidase inhibitor is a member selected from the group consisting of phenelzine, tranylcypromine, isocarboxazid, and combinations thereof.

14. A method as in claim 1, wherein the second antidepressant agent is a tricyclic antidepressant.

15. A method as in claim 14, wherein the tricyclic antidepressant is a member selected from the group consisting of amitriptyline, amoxapine, clomipramine, desipramine, doxepin, imipramine, nortryptyline, protriptyline, trimipramine, and combinations thereof.

16. A method as in claim 1, wherein the second antidepressant agent is a tetracyclic antidepressant.

17. A method as in claim 16, wherein the tetracyclic antidepressant is a member selected from the group consisting of maprotiline, mirtazapine, and combinations thereof.

18. A method as in claim 1, wherein the second antidepressant agent is a member selected from the group consisting of bupropion, duloxetine, nefazodone, trazodone, venlafaxine, and combinations thereof.

19. A method of lessening an adverse side effect that is experienced by a subject in association with taking an antidepressant drug, comprising co-administering therapeutically effective amounts of a DHEA congener and the antidepressant drug to the subject.

20. A method as in claim 19, wherein the adverse side effect associated with an amount of the antidepressant drug is less severe than the adverse side effect associated with administering the same amount of antidepressant drug alone.

21. A method as in claim 19, wherein the adverse side effect is a member selected from the group consisting of sexual dysfunction, nausea, headache, nervousness, insomnia, dry mouth, diarrhea, and anorexia.

22. A composition for the treatment of depression in a subject, comprising:
a DHEA congener;
a second antidepressant agent; and
a carrier,
wherein the composition is more effective in producing an anti-depressive effect compared to the administration of either the DHEA congener or the second antidepressant agent alone at their respective dosages.

23. A composition as in claim 22, wherein the composition is a liquid solution or suspension.

24. A composition as in claim 22, wherein the composition is a solid.

25. A composition as in claim 22, wherein the second antidepressant agent is a selective serotonin reuptake inhibitor.

26. A composition as in claim 25, wherein the selective serotonin reuptake inhibitor is a member selected from the group consisting of citalopram, escitalopram oxalate, paroxetine, fluoxetine, fluvoxamine maleate, sertraline, and combinations thereof.

27. A composition as in claim 22, wherein the second antidepressant agent is a monoamine oxidase inhibitor.

28. A composition as in claim 27, wherein the monoamine oxidase inhibitor is a member selected from the group consisting of phenelzine, tranylcypromine, isocarboxazid, and combinations thereof.

29. A composition as in claim 22, wherein the second antidepressant agent is a tricyclic antidepressant.

30. A composition as in claim 29, wherein the tricyclic antidepressant is a member selected from the group consisting of amitriptyline, amoxapine, clomipramine, desipramine, doxepin, imipramine, nortryptyline, protriptyline, trimipramine, and combinations thereof.

31. A composition as in claim 22, wherein the second antidepressant agent is a tetracyclic antidepressant.

32. A composition as in claim 31, wherein the tetracyclic antidepressant is a member selected from the group consisting of maprotiline, mirtazapine, and combinations thereof.

33. A composition as in claim 22, wherein the second antidepressant agent is a member selected from the group consisting of bupropion, duloxetine, nefazodone, trazodone, venlafaxine, and combinations thereof.
